(19) United States
(12) Patent Application Publication
(10) Pub. No.: US 2004/0243166 A1
(43) Pub. Date: Dec. 2, 2004

(54) Surgical instrument for placing a urinary incontinence pad in the lower abdomen of patients

(76) Inventors: Erich K. Odermatt, Schaffhausen (CH); Christine Weis, Tuttlingen (DE); Werner Woelfle, Duerrheim (DE); Hansjoerg Melchior, Kassel (DE)

Correspondence Address: NATH & ASSOCIATES, 1030 15th STREET, NW, 6TH FLOOR, WASHINGTON, DC, 20005, US

(21) Appl. No.: 10/485,513

(22) PCT Filed: Aug. 1, 2002
(86) PCT No.: PCT/EP02/08574

(30) Foreign Application Priority Data
Aug. 3, 2001 (DE) 101 38 955.8

(51) Int. Cl. A61B 17/34

(52) U.S. Cl. 606/185; 606/151

(57) ABSTRACT

The invention relates to a surgical instrument (1) for emplacing a urinary-incontinence strap (2) in the lower abdomens of patients, in particular, female patients, having at least one curved shaft (3) for penetrating the lower abdomen, a hand grip (8) for the shaft (3) associated with the shaft, and at least one securing device (7) for attaching the strap to the shaft.

The invention relates to a surgical instrument for emplacing a urinary-incontinence strap in the lower abdomen of patients, in particular, female patients.

Various operative methods and specially designed operating utensils and implants have been developed for combating urinary incontinence. Several surgical methods require opening the abdominal cavity in order to introduce items, similar to prostheses, that either encircle the urethra in the vicinity of the neck of the bladder or press against it from above or below (cf. WO 00/18319, WO 90/01016, WO 91/00069, U.S. Pat. No. 4,709,690, and WO 85/02993). The minimally invasive introduction of an incontinence strap in the form of a U-shaped noose that reaches around the underside of the urethra, where the free ends of the noose are either attached to the abdominal wall, for example, are sewn thereto, or terminate on the abdominal wall, without being initially attached thereto, and become anchored thereto by the ingrowth of connective tissue, that, for example, in the case of female patients, is emplaced in the abdominal cavity by inserting it through the wall of the vagina, has particularly proven its worth. Surgical instruments and such straps are described in WO 90/03766, WO 96/06567, WO 97/13465, and WO 2001/030246.

The subject matter of the invention is a surgical instrument for emplacing a urinary-incontinence strap in the lower abdomen of patients, in particular, female patients, having at least one curved shaft for penetrating the lower abdomen, a hand grip for the shaft associated with the shaft, and at least one securing device for attaching the strap to the shaft. The shaft may be configured in the form of a needle and have a circular cross-section. However, the shaft may also be configured such that it has a noncircular cross-section, in particular, a flat cross-section or a cross- section that varies over its length, a matter that shall be taken up later. The shaft may be provided in various lengths in order to adapt it to suit patients' bodily dimensions and corpulence. As a rule, the shaft is configured such that it may be attached to, and subsequently detached from, the grip, in particular, may be attached to the grip such that it is secured against axial and rotational displacements. This will allow both attaching various shafts to the grip and reusing the grip with shafts that are intended for a single use only. The shaft may be uniformly or symmetrically curved. However, it will be preferable to provide that the shaft is asymmetrically curved, which will allow safely inserting the shaft between the bladder and pubic bone. The curvature of the shaft may be configured according to whether the shaft is to be inserted along a path extending from the abdominal cavity to the urethra, or a path running in the opposite direction. According to the invention, the shaft is preferably asymmetrically curved such that it may be inserted into patients' lower abdomens from either their abdominal cavity or their crotch.

The shaft may have insertion tips for penetrating the lower abdomen on both ends or be configured to allow attaching such thereto. This will allow the operator to choose either end thereof for insertion into patients' bodies. The shaft is preferably configured such that either end thereof may be attached to the grip and subsequently detached therefrom. This will allow alternatively using both ends of the shaft and attaching the grip to that end of the shaft that protrudes from the body, after removing a detachable tip, if present, after the shaft has been inserted into the lower abdomen and withdrawing the shaft from the body using the grip, while simultaneously introducing the strap. Both ends of the shaft are preferably also configured for attaching the strap, which will provide a wide variety of operative options.

In the case of one embodiment of the invention, the securing device for attaching the strap to the shaft is configured and located such that the strap cannot be attached to the shaft until the grip has been detached from the shaft. This will guarantee that the strap can only be attached to the shaft if the shaft has already been inserted into the body and the grip has been detached therefrom. This will prevent the strap from becoming contaminated when the shaft is inserted into the body. The strap may also be atraumatically and gently emplaced. The same devices may be used for attaching the grip and the strap. However, different devices may also be used, and they may be provided at essentially the same locations in order that alternative opportunities for attaching the grip and the strap will be precluded.

In the case of another embodiment, as mentioned above, it may, for hygienic reasons, be provided that the shaft may be used once only and then discarded. Irrespective thereof, however, it will be both beneficial and desirable if each end of the strap is allocated to its own shaft, i.e., if using the same shaft for attaching the ends of the strap twice during an operation is precluded. The shaft is thus preferably equipped with a safety device that will preclude reuse of the shaft, at least during one surgical session.

The end of the strap may be attached directly to the shaft, for example, the end of the strap may be capable of being inserted into an eye on the shaft, or the end of the shaft may be configured in the form of a hook or snap link and engage an end of the strap configured in the form of a noose or provided with a hole. The strap is preferably attachable using a coupler fastened to the end of the strap, where, in particular, the end of the shaft and the coupler are configured in the form of mating plug-and-socket connectors. In order to preclude reuse of the shaft, the end of the strap and, in particular, the coupler attached to the end of the strap, may be unreleasably attached to the shaft. Once the strap has been introduced into the lower abdomen, the protruding portion of the strap may be cut off, while the end of the strap remains on the shaft. The strap may be attached to the shaft, either directly or via the coupler, using a self-locking, latching coupler. If the latching coupler is unreleasably attached to the shaft or may only be detached therefrom using a separate tool, reuse of the shaft, at least its reuse during an operation, will be precluded.

The tip of the shaft is preferably configured such that it is blunt, rather than sharp. This will help avoid injuries to abdominal organs. The insertion and withdrawal points on the body may be opened using instruments, for example, a scalpel, that have been designed for that purpose. The operator may guide the shaft through the body by using a finger to locate the shaft by feel, and then withdrawing it.

The shaft is preferably configured such that it may be inserted into the grip. The grip may have detents, preferably flexing detents, that may be locked at their detenting positions in order to axially retain the shaft in recesses in the grip. The detents may be locked using a releasable locking mechanism, in particular, a locking slide on the grip, where the locking slide is preferably at least held in position at its locking position, in particular, is held in position at both of its terminal positions, by friction. In the case of this embodiment, the end of the shaft may be inserted past the flexing detents, into the grip, withdrawn therefrom, and locked in place in the inserted position. In the case of another embodiment of the invention, the detents on the grip are configured such that they are self-locking and interact with a release member on the locking slide that is preferably held in place at the locking position by means of spring loading. In the case of this embodiment, the detents automatically lock once the shaft has been inserted into the grip, which eliminates need for a separate locking mechanism. The shaft cannot be withdrawn until the detents have been released. The shaft may also be attached to the grip axially, in particular, by means of an axial, threaded joint. The shaft may have a thread, in particular, an external thread, on that end thereof that is to be inserted into the grip, where a threaded member in the grip has a mating counterthread.

Securing the shaft against rotation while it is in the grip is preferably accomplished by providing that the shaft has a noncircular, in particular, a flattened or angular, cross-section in the vicinity of that end thereof that is to be inserted into the grip.

Suitably formed contacting surfaces on the grip will then prevent rotation of the shaft. A suitably tight fit, combined with suitably long contacting surfaces, will prevent the shaft from rocking in the grip and thus allow obtaining a wobble-free union of shaft and grip.

In the case of one embodiment of the invention, the shaft is configured in the form of a hollow bar, in particular, a tube, that is closed at least along its sides, which will allow arranging the strap inside the shaft in order that the shaft will serve as a protective enclosure for the strap during insertion into the lower abdomen. The shaft may be open on one end, in particular, on that end opposite the end thereof that is to be inserted into the grip, in order that the strap may be held in place while the shaft is withdrawn from the body.

As a rule, the shaft is solid, which will allow inexpensively manufacturing it, as well as configuring it such that it has a small cross-section, which will, in turn, also allow keeping the cross-section of the incision channel in the body as small as possible. The shaft may, as is usually the case, have a circular cross-section. However, in the case of special embodiments, the shaft will have a noncircular cross-section, which, in particular, will allow accommodating the lateral extension of the strap. For example, in the case of one embodiment of the invention, the shaft is flat over at least part of its length, preferably over its entire length. The width of the shaft may essentially equal the width of the strap over at least the broadened portions of its length, preferably over its entire length. If necessary, this will allow a blunt broadening of the channel through the tissue due to the blunt, lateral edges of the shaft, which will, in turn, guarantee that a predetermined orientation of the strap within the body will be maintained. Twisting or rolling up of the strap during insertion into the body may be prevented in this manner. The cross-section of the shaft may be flattened, oval, or elliptical. A rod having a circular cross-section may also be broadened by forming lateral extensions thereon. The rod may also be broader than the width of the strap over at least certain sections thereof, which will be beneficial, particularly in the case of adipose patients. Any transitions in its width should preferably be gradual. In the case of one embodiment, the flattening is normal to the plane of curvature of the shaft. In the case of another embodiment, the flattening lies in its plane of curvature.

The strap is beneficially attached to the shaft such that it cannot twist. This will allow predetermining the orientation of the strap within the body. The strap may preferably be attached to the shaft such that the plane of the strap lies in the plane of flexure and/or the plane of a flat side of the shaft. This will provide that the strap will come to rest in a flat orientation beneath the urethra and may be guided all the way through to the abdominal wall without twisting. Flat orientation of the strap may be predetermined in the case of a needle having a circular cross-section as well. For example, the end of the strap may be prebent into a V-shape or U-shape in the vicinity of the end of the shaft in order that it will automatically take on a flat shape over the remainder of its length. It may also be folded over once or multiply Z-folded, which will provide that it will automatically take on a flat shape over the remainder of its length.

The shaft and grip may consist of stainless steel. However, the grip may also be fabricated from plastic, which is preferable. The shaft may also partially consist of plastic, or consist entirely of plastic.

In the case of another embodiment of the invention, the shaft and grip are unreleasably joined to one another, in particular, form a single, monolithic unit. The device for reattaching the strap to the free end of the shaft is provided in the vicinity of the tip of the shaft, particularly in the case of this embodiment. In the case of this embodiment, the strap may be transported along with the shaft when the shaft is inserted into the body. It will also be feasible to poke the shaft through from one side and then attach the strap to the protruding end of the shaft and withdraw the strap, along with the shaft. The embodiment having a permanently joined shaft and grip will be particularly beneficial in cases where reusability is of great importance. Such an embodiment may be configured to have a readily cleanable surface by avoiding undercuts and other difficult-to-access spots where contaminants might accumulate.

According to the invention, it may be provided that the shaft is enclosed in a flexible sheath, from which the shaft may be withdrawn, in particular, may be withdrawn when the strap is pulled through the sheath. Such a flexible sheath may consist of resorbable material and be designed to temporarily remain in the body. The flexible sheath may also be designed for attaching and/or accommodating the strap, or may be designed to be itself an incontinence strap.

The strap may be attached inside the body by means of sewing. As a rule, textured surfaces and/or ragged edges will be sufficient to allow self-attachment. The incontinence strap is itself designed to be flexible. It may be a textile strap, in particular, such that is coated or impregnated. It may also be a strap in the form of a foil that is smooth over at least portions of its length. It may beneficially be configured from materials and/or have structures that vary over its length. This is of particular importance to its longitudinal midsection, which is intended to be arranged in the vicinity of the urethra. The urethra is particularly sensitive to irritations, which is why the strap should have surfaces that are as smooth as possible in that vicinity. The strap may also at least partially consist of resorbable material. For example, its longitudinal midsection, which is intended to be arranged in the vicinity of the urethra, may consist of resorbable plastic. It will thus be feasible to cause growth of connective tissue that will yield the desired lifting. of the urethra by making a suitable choice of resorbable material and/or structure the strap, for example, adding pores, at that location. The incontinence strap will then have served its purpose, and will no longer be needed. The remaining portions of the strap may consist of nonresorbable materials and remain in the body. The resorbable materials employed should preferably be of nonbiological origin in order prevent infections and defensive immunoreactions. Known polymers and copolymers of lactide, glycolide, trimethylene carbonate, dioxanone, and ε-caprolactone will be suitable for this purpose. Also particularly suitable is polyvinyl alcohol, whose solubility and resorption rate may be influenced by chemical modification and/or physical treatment (causing formation of crystallites).

The strap may also be configured from several, long, essentially parallel strips having differing resorbabilities for the purpose of extracting foreign matter as soon as it is no longer needed. Variations, particularly along the longitudinal direction, in particular, variations concerning its longitudinal midsection, may be caused by its mechanical construction. For example, its longitudinal edges may, with particular benefit, be smooth there, as can, preferably, its entire longitudinal midsection. A closed, in particular, smooth, surface may be provided over this longitudinal section. A width and/or thickness exceeding those elsewhere along its length may also be provided there.

In the case of a special embodiment of the invention, the thickness of the strap may be varied, in particular, may be varied over its midsection. The strap may be double-walled, preferably tubular, particularly over its longitudinal midsection. It may have a chamber, in particular, a chamber located in its longitudinal midsection, that may be filled with a fluid. The thickness and/or width of that chamber may vary with the extent to which it has been filled. The chamber may thus at least partially consist of elastic, stretchable, material. The walls of the chamber that may be filled with fluid may at least partially consist of a pierceable, self-sealing material, in particular, silicone rubber, which will allow subsequently altering the size and shape of the chamber by filling it or drawing off fluid therefrom. The chamber that may be filled with fluid may also be provided with a fluid line extending at least to the end of the strap. Such a chamber and a fluid line connected thereto may, in the case of a thermoplastic material, be configured by welding the walls of tubing having the desired contouring. The strap beneficially has a crinkled surface and/or porous structure that will promote self-anchoring onto the abdominal wall on those sections thereof that are intended to be arranged on the abdominal wall. It will be beneficial if both ends of the strap are configured for attachment to a shaft, in particular, a curved shaft, used for emplacing the strap within the abdominal cavity. The ends of the strap may be reinforced for that purpose. The ends of the strap may be narrower than the remainder of the strap. The ends of the strap may also be provided with a hole.

It may also be provided that the strap is arranged in a sheath, preferably a sheath that is transversely split at its midsection, or may be split there, that may be withdrawn following implantation of the strap. The sheath serves to both protect the strap, in particular, protect it from contamination. On the other hand, the sheath may ease insertion of the strap if it has a rough outer surface. The strap may be configured such that it may be separated along the longitudinal direction by, for example, configuring it from overlapping strips. The ends of the strap may also be allocated to securing devices, with which the ends of the strap may be attached to the abdominal wall in order to, for example, prevent the ends of the strap from sliding toward one another when the sheath is withdrawn.

Other features of the invention will be evident from the following descriptions of embodiments of the invention, together with the figures and subordinate claims. The individual features involved may be configured either singly, or in the form of combinations of several such with one another.

The figures depict:

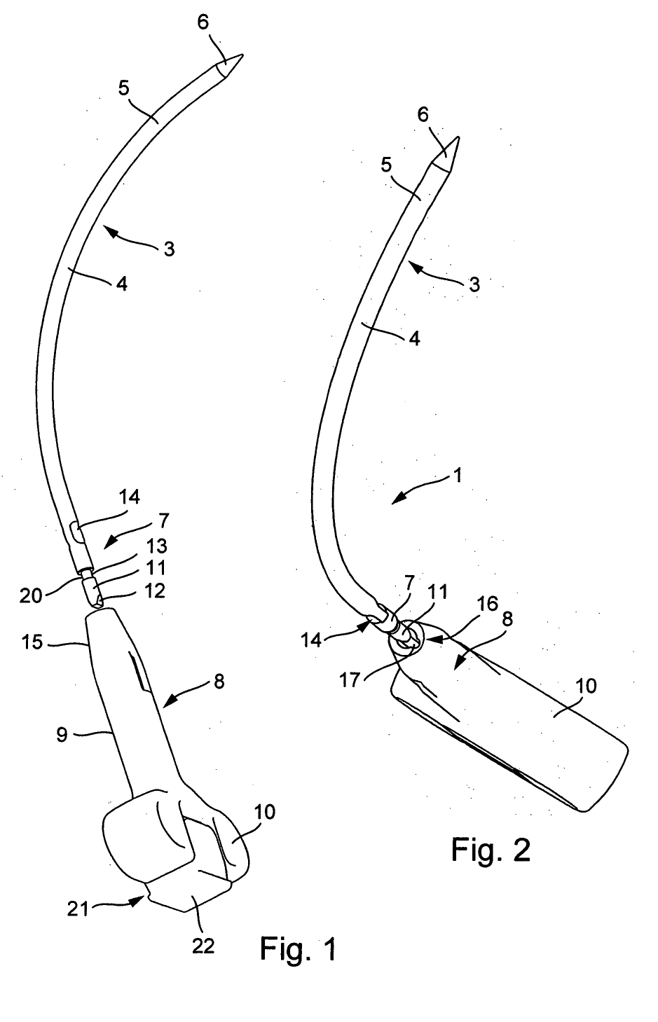

FIG. 1: a perspective view of an embodiment of an instrument for inserting an incontinence strap, where the instrument has a curved shaft and a handling grip for the shaft, and is shown here with the shaft and grip separated;

FIG. 2: the embodiment shown in FIG. 1, shown here in another perspective view;

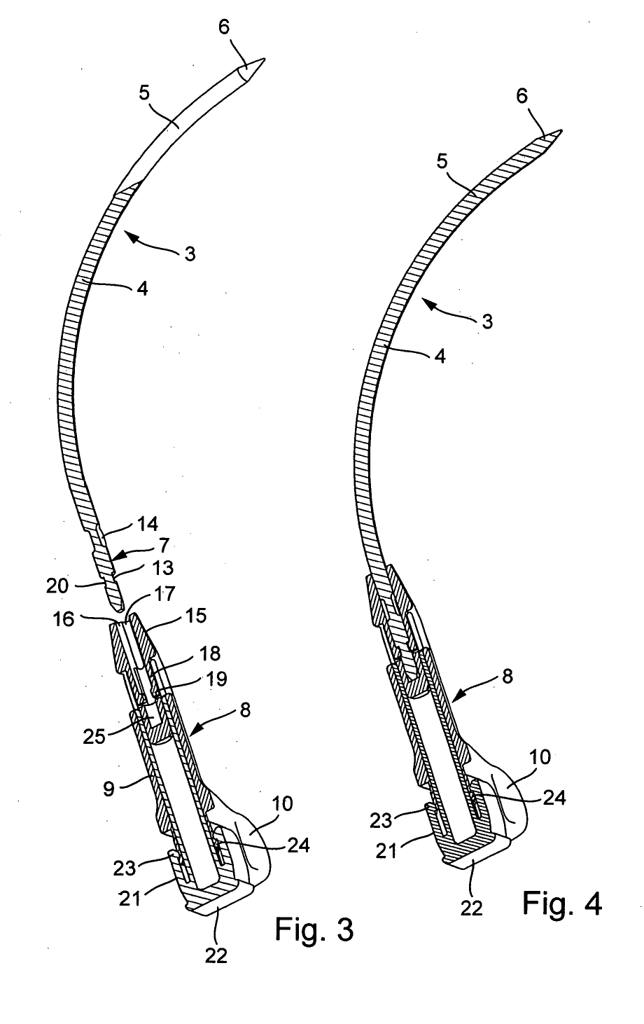

FIG. 3: a sectioned view of the same shaft and handling grip shown in FIG. 1;

FIG. 4: a longitudinal section through the shaft and grip, where the grip has been inserted onto the shaft, but has not yet locked onto the shaft;

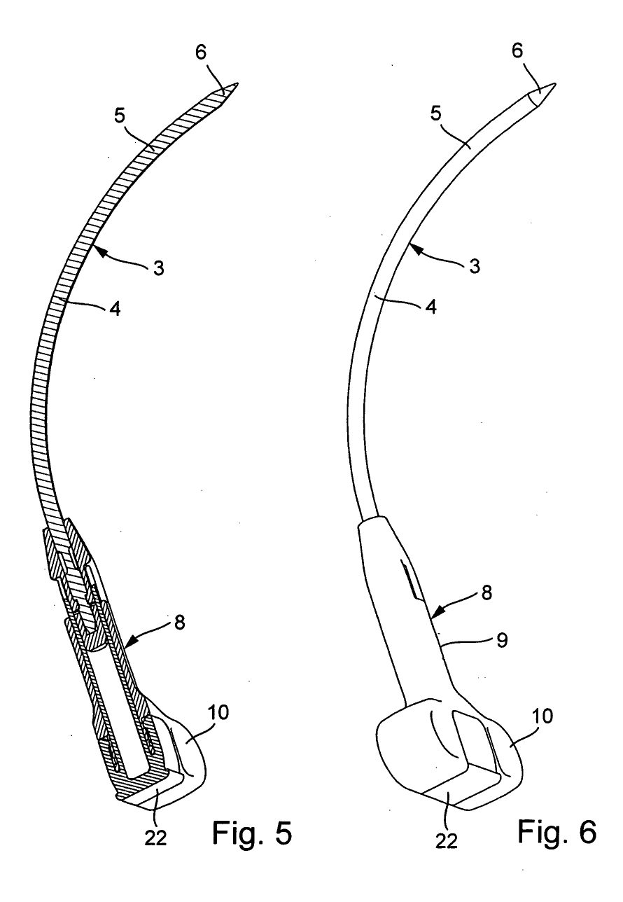

FIG. 5: a longitudinal section through the shaft and grip in the locked state;

FIG. 6: a perspective view of the shaft and grip in the locked state;

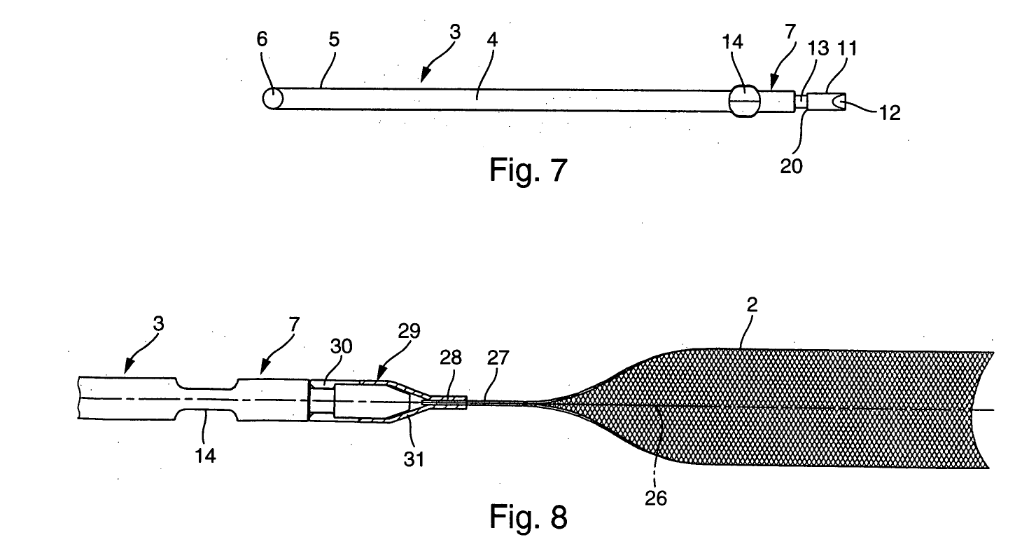

FIG. 7: a view of the shaft, normal to the plane of curvature of the shaft;

FIG. 8: a view of part of the shaft and an incontinence strap attached thereto;

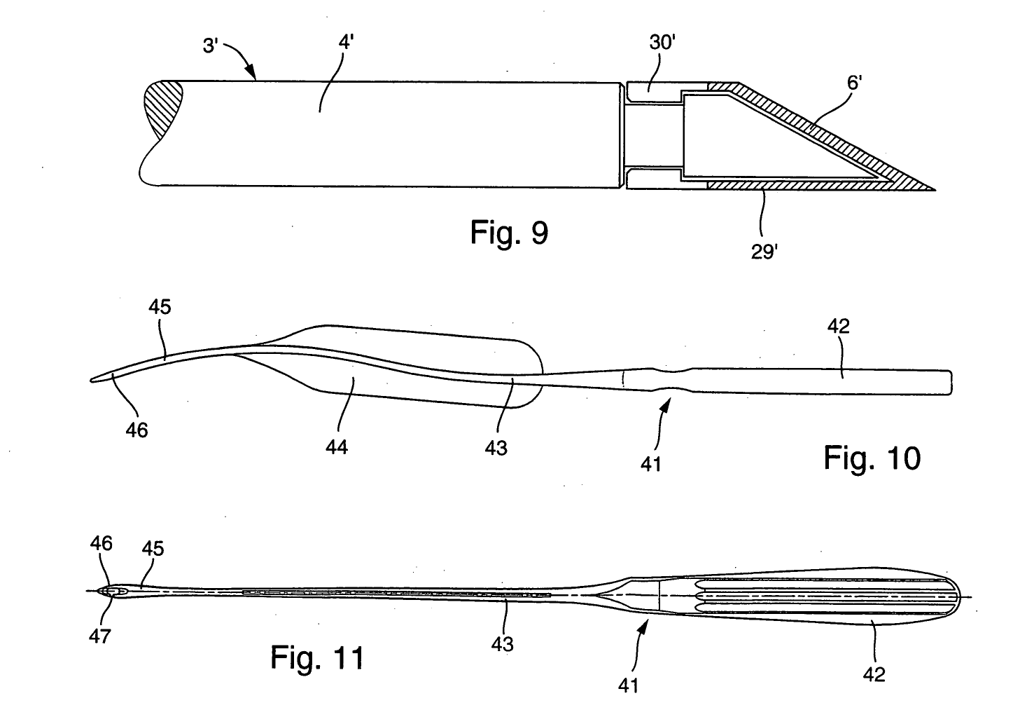

FIG. 9: a view of one end of a shaft of another embodiment that may alternatively be attached to a grip, have an incontinence strap attached to it, or have a tip inserted onto it;

FIG. 10: a side view of another embodiment of an instrument for inserting an incontinence strap;

FIG. 11: a top view of the same embodiment shown in FIG. 10;

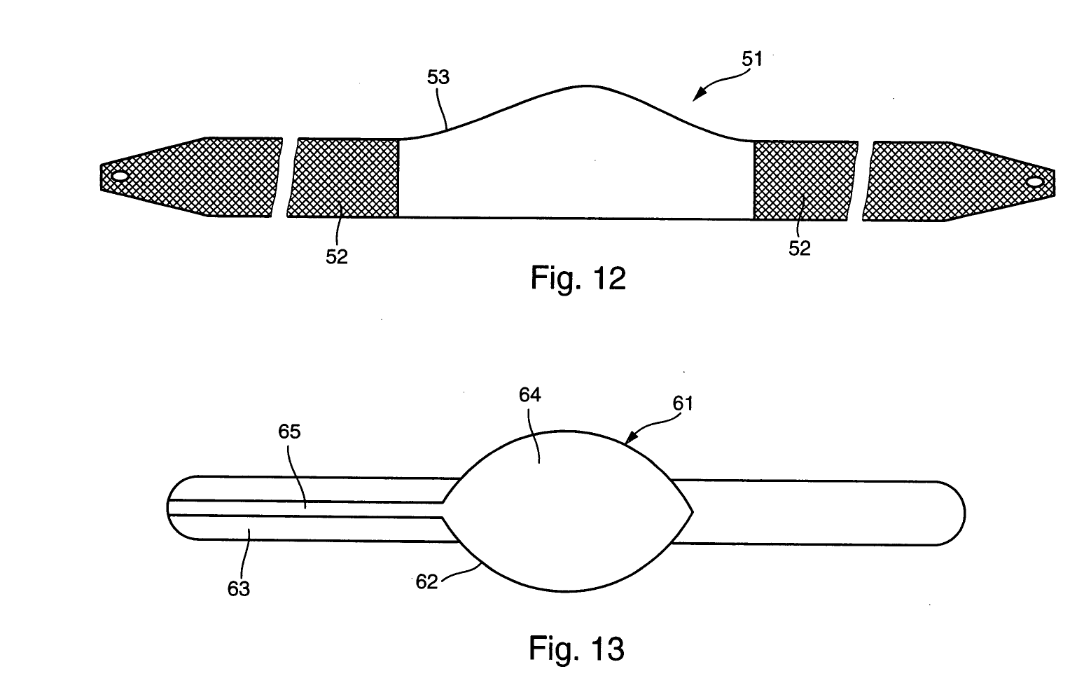

FIG. 12: an embodiment of an incontinence strap; and

FIG. 13: another embodiment of an incontinence strap.

In the case of the embodiment of an instrument 1 for emplacing an incontinence strap 2 shown in FIGS. 1-9, a shaft for inserting the incontinence strap is provided in the form of a curved, solid, stainless-steel needle 3 in the form a curved, circular rod 4. The needle has a conical tip 6 on an insertion end 5. This may also be ground off at an angle such that the edge of the ground-off section lies on the inner side of the curvature. The tip of the needle is not sharp in order to prevent injuries to the abdominal organs when the incontinence strap is inserted. Since the incisions in the abdominal wall and vaginal wall will, in any case, be made using other instruments, such as scalpels, the needle essentially acts as a guiding appendage only. The end of the needle opposite that bearing the tip 6 is configured in the form of an attachment end 7 and has devices for attaching it either to a grip 8, or to the incontinence strap 2.

The grip 8 consists of plastic or steel and has a longitudinal section 9 that is essentially configured in the form of a hollow cylinder that is used both for accommodating and attaching the end 7 of the needle, and for gripping with the hand. The rear end of the grip has a broadened section 10 that essentially gives the grip a T-shape and is also used for gripping, and allows precisely controlled, lateral displacements of the needle.

As may be seen from FIG. 7, the attachment end 7 of the needle 3 has a cylindrical section 11 having a diameter less than the normal diameter of the needle that has a beveled surface 12 on one side, or either side, and is separated from the circular rod 4 of the needle by a circumferential groove 13 that forms an intervening cylindrical section having an even smaller diameter. This end of the needle is also is provided with an antirotation lug that has a cross-section differing from the circular cross-section of the needle, in particular, an antirotation lug in the form of a stamped flat 14, in the vicinity of that section of the needle where the needle has its full diameter.

The grip 8 is symmetric with respect to the central plane of intersection orthogonal to the plane of the “T” and has in its interior a receptacle for holding the needle. The needle will not be free to rotate, and the grip has on its conically tapered attachment end 15 an insertion bore 16 whose inner diameter equals the normal diameter of the needle, and also has a pair of lateral, flat-bottomed, longitudinal grooves 17 whose dimensions equal those of the stamped flat 14 on the needle and guarantee seating of the needle such that it will not be free to rotate and will be in the correct orientation with respect to the grip. When in that orientation, the plane of curvature of the needle is orthogonal to the plane of the T-shaped grip.

The walls of the hollow, cylindrical, longitudinal section 9. of the grip have a pair of thinner sections formed by mirror-image, with respect to the plane of the “T,” window-like recesses on diametrically opposite sides that are configured in the form of detenting prongs 18. These detenting prongs 18 are longitudinal leaf springs whose end facing the insertion bore of the grip forms a unit with the material of the longitudinal section 9 and whose free end has a detenting protuberance 19 that faces inward. As shown in FIG. 4, the detenting prongs 18 latch into position with their detenting protuberance 19 situated in the circumferential groove 13 on the end 7 of the needle, but are not self-locking. On the contrary, their detenting protuberances 19 have flats on their sides facing toward, and away from, the direction of insertion and may be flexed and spread apart by the beveled surfaces 12 on the end of the needle aligned on them when the end 7 of the needle is inserted and by the shoulder 20 on the circumferential groove 13 when the end 7 of the needle is withdrawn. The detenting prongs are locked by a slide 21 in the form of a sleeve arranged within the grip that may be slid along the longitudinal axis of the grip that engages the outer surfaces of the detenting prongs 18 when in its locking position (cf. FIG. 5) and rests with its engaging, free end 22 abutting against the broadened section 10 on the end of the grip, where it self-locks onto the housing of the grip by flexing. In the disengaged position (cf. FIGS. 3 and 4), the slide 21 releases the detenting prongs 18 and overlaps the end of the grip. Detenting elements 23 on the slide, together with associated active elements 24 on the grip, prevent the slide from falling off the grip when in its disengaged position. The grip may be disassembled for cleaning, i.e., the slide 21 may be slid over the active elements 24 on the grip 8 and off the grip in order to remove it. The interior of the grip also has a hollow section 25 that has an inner diameter that is less than that of the grip and equals the outer diameter of the cylindrical section 11 on the end 7 of the needle, and serves as a receptacle for that cylindrical section. This guarantees wobble-free seating of the needle in the grip.

The strap 2 is formed from a flat strip of textile netting. Both ends of the strap are folded along the centerline 26 of the strap, yielding end sections 27 that are half as wide as the remainder of the strap, and whose planes are orthogonal to the plane of the strap. These end sections 27 are firmly clamped in the flattened ends 28 of a retaining sleeve 29 on the end 7 of the needle that fits on the cylindrical section 11 thereof having a smaller diameter and latches into position thereon. The retaining sleeve has the same outer diameter as the circular rod 4 of the needle. The free end 30 of the retaining sleeve 29 is longitudinally slit, where the tips of the prongs formed thereby are either bent inward or configured such that they are thicker, snap into place in the circumferential groove 13 on the end of the needle, and reach around, and abut against, the circumferential shoulder 20, in order that it may be attached to the end 7 of the needle. The strap 2 may be attached to the needle 3 such that it cannot twist, in particular, is attached thereto oriented such that the plane of the strap lies in the plane of curvature of the needle. The single flat 12 (cf. FIG. 7) or pair of flats on the cylindrical section 11 of the needle interact with the sloping surfaces 31 formed by the stamped flats on the retaining sleeve 29 for that purpose. However, other configurations of the antitwisting mechanism are feasible.

In the case of the embodiment of the invention shown, the means for attaching the strap may be such that they prevent successively attaching opposite ends of the strap to the same needle, i.e., prevent the same needle from being used twice during an operation. The retaining sleeve 29 on the end 7 of the needle is thus either unreleasably attached thereto, or may only be removed therefrom using a tool that has been specially designed for that purpose. This will preclude attachment of the other end of the strap to the end 7 of the needle and prevent reuse of the needle during the same surgical session. In the case of an unreleasable attachment, the needle 3 is discarded, after cutting off the strap 2, together with the retaining sleeve 29. If the needle 3 is to be reused, the retaining sleeve 29 may be removed from the needle using the special tool, prior to cleaning and sterilizing the needle.

It may also be provided that the needle 3′ has tips 6′ on both ends by, for example, providing that its attachment end 7 is also pointed or has been ground off an angle. Alternatively, it may also be provided that tips for the needle are configured such that they may be attached to the needle 3′, in particular, to the circular rod 4′, as shown in FIG. 9. Both ends of the needle may be configured such that tips 6′ may be attached thereto. For example, both ends of the needle may be configured similarly to its attachment end 7′. A sleeve 29′ having a tip 6′, instead of a retaining sleeve, may then be slipped onto the needle. It may also be provided that the tips are designed to be detachable and the retaining sleeve attached to the strap may be slipped onto the needle once the tips have been detached.

The instruments shown in FIGS. 1-9 have a wide variety of applications due to the opportunities for altering their setups that they provide, and may be used in surgery in which their shaft is inserted through the abdominal wall from above and guided downward, or inserted from below, for example with female patients through the vaginal wall and guided upward to the abdominal wall. The strap may be pulled through by the needle, as in the case of suturing, by guiding the needle through the lower abdomen and allowing the needle to transport the strap along with it. The strap may also be picked up by, for example, guiding the needle downward from the abdominal wall, attaching the strap to the accessible tip of the needle, and withdrawing the needle, along with the strap.

The embodiment of an instrument for emplacing an incontinence strap shown in FIGS. 10 and 11 has fewer prospective applications than the aforementioned instruments. However, the instrument 41 shown there is more simply constructed and has several special features. The surgical instrument 41 has a grip 42 and a shaft 43 that are configured in the form of a single, monolithic unit and beneficially consist of stainless steel. The grip 42 is long and flat, but may also have another shape suitable for use on surgical instruments. The shaft 43 follows a gentle S-curve. It has a adjacent to the grip 42 broadened section 44 in the form of a flat rod having blunt, parallel, lateral edges terminating in a curved section 45 having an essentially circular cross-section that has a hook 47 opening sideward for attaching an incontinence strap to its tip 46. The transition from the broadened section 44 to the narrow section 45 is gradual, and achieved by tapering the broadened section. The plane of the broadened section lies in the plane of curvature of the curved section 45. The lateral edges of the broadened section 44 curve slightly upward relative to those of the grip 42, and those of the curved section 45 on which it terminated curve slightly downward relative thereto. However, the shaft may also be configured in the form of a curved rod having an essentially circular cross-section.

The embodiment according to FIG. 10 is intended for use in cases where the shaft is guided through the abdominal wall and downward, into the abdominal cavity. The purpose of the broadened section 44 is giving the incision channel through the abdominal wall and the connective tissues and fatty tissues lying thereunder, extending downward to the vaginal wall, a broadened shape that will roughly correspond to that of the incontinence strap. The broadening may essentially extend over the entire length of the shaft. Once the shaft has been inserted into the abdominal cavity, the strap may be attached to the tip 46 of the shaft 43, which has been guided through from below, for example, guided through the vaginal wall, and withdrawn along with the instrument when the instrument is withdrawn. In the case of two incisions in the abdominal wall, to the right and left of the center thereof, or, preferably, a central incision above the pubic bone, the ends of the strap on the right and left of the urethra, respectively, may be pulled upward, yielding a U-shaped noose reaching around the underside of the urethra.

The tips on the shaft may be configured such that they will allow attaching the strap such that it may be subsequently detached, in particular, may be configured such that they provide an attachment mechanism having a securing device that will prevent the strap from becoming unintentionally detached from the shaft. In the case of the embodiment shown, the tip 46 on the shaft is preferably configured in the form a so-called “Reverdin needle”. It has an eye that may be opened and closed and has a hooked shape when in the opened state. However, the attachment mechanism on the tip 46 may also be configured differently, for example, configured in the form of a simple eye, or by configuring the tip in the form of a detachable cap and providing that a coupling element, to which a mating coupling element on the strap may be attached, will be exposed when the tip is removed, as has been described in conjunction with FIG. 9. The eye may also be closed by means of a cap nut.

In the case of an embodiment of an incontinence strap according to FIG. 12, the incontinence strap 51 has longitudinal sections that differ from one another. Two end sections 52, each about 10 cm to 20 cm long and about 2 cm wide, are configured in the form of coarse-meshed textile straps that have rounded or tapered, V-shaped ends and holes, or other attachment facilities, for attaching them to an insertion instrument. A midsection 53 consists of resorbable plastic and is broadened, compared to its end sections 52, on one side by a sort of curved extension. This section consists of smooth foil material, has smooth edges. The section is intended to reach around the underside of the urethra. Its smooth contours, and the fact that it has been designed to protect the urethra, will help prevent injuries to the urethra due to undesired, unrestrained proliferation of newly formed, connective tissue. Insertion of this section of the strap, which represents a foreign object, will provide that sufficient tissue for supporting the urethra from below will be formed. Since the implant will thus have served its purpose, this particular section of the strap may be configured from resorbable material.

The degradation period and resorption time of the material used for configuring the midsection may be predetermined in a known manner employing suitable copolymerization processes. The polymers that come into question are synthetic polymers, in particular, such that may be obtained by polymerizing lactide, glycolide, trimethylene carbonate, dioxanone, or ε-caprolactone, or mixtures thereof.

In the case of the embodiment shown in FIG. 13, an incontinence strap that is intended to remain in the body indefinitely is provided. Once again, the incontinence strap 61 is subdivided into various sections, where at least one midsection 62 is configured in the form of a chamber that may be filled with a fluid, for example, water or liquid contrast media. The midsection 62, or even the entire strap, may be configured in the form of a tube for that purpose. Various types of constructions and materials are available for configuring the strap. In the case of the embodiment shown in FIG. 13, at least the strap's midsection and at least a channel section 63 bordering on its midsection consists of thermoplastic material. Seam-welding opposing walls of the tubing will allow configuring the chamber 64 and the communicating fluid channel 65 extending along the channel section 63, through which the chamber may be filled and the fill-volume of the chamber may be subsequently corrected whenever necessary. The fluid channel 65 may be closed, for example, welded closed, once the chamber has been filled. It may also be accessible beneath the abdominal skin via a valve, for example, a pierceable membrane, that is accessible from outside.

The chamber may also be formed from an elastomer, for example, silicone rubber, that is pierceable and self-sealing. In this case, no separate liquid channel will be required. It will also be feasible to configure the entire strap in the form of a tube and merely seal both ends. The only essential feature is that strap, complete with the chamber that may be filled, be in the form of a strip, which will allow the minimally invasive emplacement of the strap, without opening of the abdominal cavity being required.

1. A surgical instrument (1, 41) for emplacing a urinary-incontinence strap (2, 51, 61) in the lower abdomens of patients, in particular, female patients, having at least one curved shaft (3, 43) for penetrating the lower abdomen, a hand grip (8, 42) for the shaft (3, 43) associated with the shaft, and at least one securing device (7, 47) for attaching the strap to the shaft.

2. A surgical instrument according to claim 1, wherein the shaft (3) is configured such that it may be attached to, and subsequently detached from, the grip (8) and, in particular, may be attached to the grip (8) such that it may secured against axial and rotational displacements.

3. A surgical instrument according to claim 1 or claim 2, wherein the shaft (3) is curved, in particular, asymmetrically curved, such that it may be inserted into the lower abdomen from either the abdominal cavity or the crotch.

4. A surgical instrument according to any of the foregoing claims, wherein the shaft has introduction tips for penetrating the lower abdomen on both ends, or may have such attached to it.

5. A surgical instrument according to any of the foregoing claims, wherein both ends of the shaft are configured such that either end thereof may be attached to the grip and subsequently detached therefrom.

6. A surgical instrument according to any of the foregoing claims, wherein both ends of the shaft are configured such that the strap may be attached to either end thereof.

7. A surgical instrument according to any of the foregoing claims, wherein the strap (2) may be attached to the corresponding end of the shaft (3) only while the grip (8) is detached from the shaft.

8. A surgical instrument according to any of the foregoing claims, wherein the shaft (3) is intended for a single use only, each end (27) of the strap is allocated to its own shaft (3), and the shaft (3) preferably has a safety device (7, 29) for precluding its reuse.

9. A surgical instrument according to any of the foregoing claims, wherein the strap (2) may be attached the shaft (3) using a coupler (29) attached to the end (27) of the strap, in particular, wherein the end (7) of the shaft and the coupler (29) are configured in the form of mating plug-and-socket connectors.

10. A surgical instrument according to any of the foregoing claims, wherein the end (27) of the strap, in particular, the coupler (29), may be unreleasably attached to the shaft (3) and the end (27) of the strap, or the coupler (29), remains on the shaft when the strap is detached therefrom.

11. A surgical instrument according to any of the foregoing claims, wherein the strap (2) may be attached to the shaft (3) using a self-locking, latching coupler (7, 20, 29, 30).

12. A surgical instrument according to any of the foregoing claims, wherein the shaft (3) is configured such that it may be inserted into the grip (8) and the grip (8) has detents (18), preferably flexing detents, that may be locked at their detenting positions and are capable of engaging recesses (13) in the shaft (3) in order to axially retain the shaft in the grip.

13. A surgical instrument according to claim 12, wherein the detents (18) may be locked using a locking slide (21) on the grip (8) that is preferably held in locked position by friction, at least at its closed position.

14. A surgical instrument according to any of the foregoing claims, wherein the cross-section of the shaft (43) is noncircular over a section that lies outside that enclosed by the hand grip (42), in particular, has an extension in a direction that preferably lies in the plane of curvature of the shaft that greatly exceeds that in the orthogonal direction.

15. A surgical instrument according to any of the foregoing claims, wherein the shaft is configured in the form of a flat, in particular, curved (flat-oval, elliptical, circular, with lateral extensions), bar over at least part of its length, in particular, over its entire length.

16. A surgical instrument according to any of the foregoing claims, wherein the shaft has a width that is approximately equal to the width of the strap to be attached thereto over at least part (44) of its length.

17. A surgical instrument according to any of the foregoing claims, wherein the shaft has a width that exceeds that of the strap over at least part of its length.

18. A surgical instrument according to claim 17, wherein any changes in its width are preferably gradual transitions.

19. A surgical instrument according to any of the foregoing claims, wherein the strap (2) may be attached to the shaft (3) such that it cannot twist, in particular, cannot twist about an axis parallel to the plane of flexure and/or the plane of the flat side of the shaft.

20. A surgical instrument according to any of the foregoing claims, at least part of which, in particular, the grip (8), consists of plastic.

21. A surgical instrument according to any of the claims 1, 8-11, or 14-19, wherein the shaft (43) and the grip (42) are unreleasably joined to one another, in particular, form a single, monolithic unit, and a securing device (47) for securing the strap is provided on the free end (46) of the shaft.

22. A surgical instrument according to claim 21, wherein the securing device has a latching device that prevents unintentional detachment of the strap.
